(19) United States
(12) Patent Application Publication
(10) Pub. No.: US 2004/0137457 A1
(43) Pub. Date: Jul. 15, 2004

(54) Method

(76) Inventor: Bent Karsten Jakobsen, Oxfordshire (DK)

Correspondence Address: BANNER & WITCOFF, 1001 G STREET N W, SUITE 1100, WASHINGTON, DC, 20001, US

(21) Appl. No.: 10/475,524

(22) PCT Filed: Apr. 30, 2002
(86) PCT No.: PCT/GB02/01979

(30) Foreign Application Priority Data
Apr. 30, 2001 (GB) 0110547.7

(51) Int. Cl. C12Q 1/68; G01N 33/53; G01N 33/567

(52) U.S. Cl. 435/6; 435/7.2

(57) ABSTRACT

The present invention provides a method for identifying an entity which binds to a peptide-Major Histocompatibility Complex (pMHC) complex. In the method, a pMHC complex is formed between a predetermined MHC and a peptide which is obtained from a protein sequence and is not known to be capable of being complexed with the predetermined MHC. The resulting pMHC is used to screen for an entity which binds to the pMHC complex.

The present invention relates to a method for identifying an entity which binds to a peptide-Major Histocompatibility Complex.

Major histocompatibility complex Class I and II proteins, (MHC, or HLA in man), bind peptide antigens and present them on the cell surface. These MHC-peptide complexes are recognised by T lymphocytes expressing a unique T cell receptor (TCR) matching the specific MHC-peptide combination. A wide spectrum of cells can present antigen, as MHC-peptide complexes, and the cells that have this property are known as antigen presenting cells (APCs). The type of cell that presents a particular antigen depends upon how and where the antigen first encounters cells of the immune system. APCs include the interdigitating dendritic cells found in the T cell areas of the lymph nodes and spleen; Langerhan's cells in the skin; follicular dendritic cells in B cell areas of the lymphoid tissue; monocytes, macrophages and other cells of the monocyte/macrophage lineage; B cells and T cells; and a variety of other cells such as endothelial cells and fibroblasts which are not classical APCs but can act in the manner of an APC.

APCs are recognised by a subgroup of lymphocytes which mature in the thymus (T cells) where they undergo a selection procedure ensuring that T cells which respond to self-peptides are eradicated (negative selection). In addition, T cells that do not have the ability to recognise the MHC variants presented, fail to mature (positive selection).

Recognition of specific MHC-peptide complexes by T cells is mediated by the TCR, which consists of an α and a β chain, both of which are anchored in the membrane. In a recombination process similar to that observed for antibody genes, the TCR α and β genes rearrange from Variable, Joining, Diversity and Constant elements creating enormous diversity in the extracellular antigen binding domains (10¹³ to 10¹⁵ different possibilities). Antibody receptors and TCRs are the only two types of molecules that recognise antigens in a specific manner. The TCR is the only receptor specific for particular peptide antigens presented in MHC, where the peptide is often the only sign of an abnormality within a cell.

CD8 and CD4 are transmembrane glycoproteins characteristic of distinct populations of T lymphocytes whose antigen responses are restricted by class I and class II MHC molecules, respectively. CD8 and CD4 play major roles both in the differentiation and selection of T cells during thymic development and in the activation of mature T lymphocytes in response to antigen presenting cells. CD8 and CD4 are therefore considered to be the main accessory molecules for T cell receptors. CD8 is characteristic of the cytotoxic T cells (CTLs) that scrutinise the body's cells for abnormal HLA-peptide complexes. If abnormalities are found the CTL is activated and kills the cell that triggered it's activation. CD4 is expressed by T helper cells that direct the activation of other immune cells in general and the B cells in particular.

There is a need for compounds which can be used in the treatment of auto-immune disorders such as rheumatoid arthritis, lupus erthymatosus, psoriasis vulgaris, ankylosing spondylitis, Reiter's disease, post-salmonella arthritis, post-shigella arthritis, post-yersinia arthritis, post-gonococcal arthritis, uveitis, amylodosis, idiopathic hemachromatosis and myasthenia gravis, as well as the prevention of graft rejection and the treatment of graft-versus-host disease. Immune suppressor compounds include suppressors of the cellular arm of the immune system, such as suppressors of CD4 or CD8 T cells.

Attempts have been made to use antibodies directed against CD4 and CD8 as suppressors of CD4/CD8 T cells (De Fazio, et al. (1996)). However, this approach has had limited success as antibodies in general are not well suited as drugs since they tend to induce secondary immune responses and are short-lived. Another approach has involved the administration of steroids to suppress the immune system. However, there are several drawbacks associated with steroid administration. The effect of the steroids in the body is indirect and steroids cannot, therefore, be targeted to a particular location (e.g.tissue, organ etc) in the body. Moreover, steroids are known to cause undesirable side-effects when administered over a period of time.

The present invention aims to provide an integrated method that facilitates the identification of compounds with a therapeutic application. Specifically, the invention aims to facilitate the identification of compounds that act against novel targets that have application in the treatment of auto-immune disorders (such as those mentioned above) and cancer-associated diseases. Although some or all of the stages in the integrated method are known the integration of these stages results in an advance in the art by providing both a generically applicable means of identifying novel peptides capable of being loaded by MHC complexes derived from disease-associated genes or proteins and also potential therapeutics capable of blocking the interaction between said pMHCs and T cells restricted by these pMHCs.

According to a first aspect of the invention, there is provided a method for identifying an entity which binds to a peptide-Major Histocompatibility Complex (pMHC) complex, the method comprising:

complexing a peptide with a predetermined MHC thereby forming a pMHC complex, wherein the peptide is obtained from a protein sequence and is not known to be capable of being complexed with the predetermined MHC; and

screening for an entity which binds to the pMHC complex.

In the present invention, the peptide (which may or may not be post-translationally modified, e.g. glycosylated) is not known to be capable of being complexed with the predetermined MHC. Thus, the peptide may be obtained from a protein which is not known to contain a peptide capable of binding to the MHC. In addition, the peptide may be obtained from a protein which is known to contain a peptide capable of binding to the MHC, but from a part of a protein which is not known to contain peptides capable of binding the MHC. Finally, the peptide maybe obtained from a part of a protein which is known to contain a known peptide capable of binding an MHC, but the peptide itself has not been shown to be capable of binding the predetermined MHC, e.g. the peptide of interest may overlap with the known peptide. Thus, as used herein and unless the context dictates otherwise, “protein sequence” includes whole proteins or parts thereof.

The protein sequence may be identified by proteomic analysis. Alternatively, it may be predicted from a nucleic acid sequence, which may be identified by genomic analysis. Genomics and proteomics allow the identification of nucleic acids and proteins respectively, the expression or characteristics of which change in a disease state. Chambers et al, (2000) and Yoshida et al, (2001) provide reviews of proteomics methods suitable for the identification of disease related proteins. Kennedy (2000), and Emilien et al, (2000) provide reviews of genomics methods suitable for the identification of disease related genes. In certain embodiments, the present invention allows the data produced by genomics/proteomics to be used to identify potential therapeutics entities. Such entities can be used in immunosuppression (e.g. by preventing a T cell response normally initiated by the pMHC complex), or in the treatment of cancer. In the latter respect, entities can be identified which bind to a specific pMHC which is associated with cancer. Accordingly, these targeted entities can be used in the treatment and/or diagnosis of cancer, e.g. in their own right or by delivering labels or cytotoxic agents.

In one embodiment, the peptide is taken from a population of peptides, all of which are obtained from the protein sequence. The population of peptides may represent all or a part of the protein sequence. In the present invention, a protein sequence may be digested into a series of peptides, and each peptide screened to determine (a) whether it can complex with an MHC, and if it does, (b) whether there is an entity which binds to the resulting pMHC. Each individual peptide may be assayed in an individual well or container. Alternatively, an antigen presenting cell may be transformed with a vector such that it expresses the protein sequence, such that the cell causes the protein sequence to be digested into a population of peptides, one or more of which may be presented by the cell.

There is a number of peptide mapping approaches, known to those skilled in the art, which could be used in the present invention for the production of a peptide library from the protein of interest. These libraries include those based on phage display, ‘peptides on pins’, ‘peptides on beads’ and free peptides. For a review of these peptide library methods see Scott (1994). In a specific embodiment, a free peptide library method will be used in the present invention.

The protein sequence may be modified relative to a native protein sequence, the population of peptides representing the part(s) of the protein sequence which is/are modified. Thus, where for example a protein sequence is modified relative to a native protein sequence in a disease state, the population of peptides may be taken from the part of a protein sequence which is modified. The, or each, modification may caused by a mutation in the nucleic acid (e.g. DNA) sequence encoding the native protein sequence.

The population of peptides may be a series of overlapping peptides, as this increases the chances of identifying a peptide which will complex with the MHC of interest.

The peptide preferably consists of the minimum sequence necessary to enable T cell receptor binding to the pMHC complex. For example, a peptide which has been identified as being able to be complexed with an MHC may be “trimmed” so as to identify the minimum sequence necessary to enable T cell receptor binding to the pMHC complex. In this regard, the “anchor residues” of the peptide can be identified.

In the present invention, the Major Histocompatibility Complex (MHC) may be a Human Leuckocyte Antigen (HLA), and may be a class I, class II or non-classical HLA molecule. However, it is to be understood that the present invention also finds utility in identifying entities which bind to pMHC of non-human animals, including mammals such as horses, dogs, cats, etc.

As mentioned briefly above, the pMHC complex may be produced by transforming cells presenting MHC molecules with a vector which expresses the protein sequence or peptide. The vector may be a virus, such as a Vaccinia virus (Border C C and Earl P L, 1999).

There are a number of alternative expression systems, known to those skilled in the art, that could be used in the current invention for the expression of the protein of interest. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids, all may be used for expression in accordance with this aspect of the present invention. Generally, any vector suitable to maintain, propagate or express a polypeptide in a host may be used for expression in this regard.

In one embodiment, the method of the present invention further comprises the step of identifying whether the pMHC complex binds to a T cell receptor, the step comprising contacting the pMHC complex with T cells or with T cell receptors under conditions suitable to allow binding between the T cell receptors and the pMHC complex and identifying whether binding between the T cell receptors and the pMHC complex has occurred. Surface plasmon resonance (SPR) based instruments such as the BIAcore 3000™ are appropriate for assays of this type utilising soluble MHCs and soluble TCRs.

A T cell which binds to the pMHC complex may be isolated. There are number of methods, known to those skilled in the art, for the isolation of T-cells. These include the use of pMHC tetramers (Ogg 2000) in which peptide loaded MHC-complexes are synthesised by the addition of biotin to the interior aspect of refolded MHC molecules, The molecules are then mixed with fluorescent labelled strepavidin and isolated using flow cytometric technology to isolate the specific T-cells. In addition, limiting dilution analysis (Carmichael 2000) may be used to isolate specific T-cells. This involves setting up multiple replicate microcultures in which the initial number of responder cells is progressively reduced over an appropriate range of low dilutions, so as to include the dilution at which there is on average one precursor cell of interest per microculture. It is preferred if the method of using pMHC tetramers to isolate T-cells from peripheral blood is used in the present invention.

There are number of methods, known to those skilled in the art, for the cloning of T-cells. These include culturing the isolated T-cells directly with irradiated EBV transformed B-cell lines and irradiated peripheral blood mononuclear cells from autologous individuals. Alternatively, T-cells can be cloned during limiting dilution analysis without bulk culture (Vyakarman 2000). Additional, antigenic-specific clones can be carried out using bulk cultures (Vyakarman 2000). Both of these methods are useful for the isolation of CD4 T-cells. It is preferred if the method using irradiated EBV transformed B-cells and peripheral blood mononuclear cells following single cell isolation is used in the present invention.

A T cell receptor specific for (i.e. which binds specifically to) the pMHC complex may be isolated. The T cell receptor may be isolated by isolating nucleic acid encoding the T cell receptor, transforming or transfecting a host cell with said nucleic acid, culturing the host cell under conditions enabling expression and isolating the expressed T cell receptor.

The T cell receptor may be a soluble T cell receptor. The soluble T cell receptor may be produced as described in WO 99/60120.

Alternatively, the method of the present invention may further comprise screening for an entity (e.g. a small organic molecule, an antibody, a receptor) which interferes with (e.g. inhibits or reduces) the interaction between the pMHC complex and a T cell or T cell receptor.

In this respect, the method of the present invention may comprise contacting a candidate entity with a T cell receptor and the pMHC complex under conditions suitable to allow interaction between the candidate entity, the T cell receptor and the pMHC complex, and determining the extent of interference by the candidate entity to the interaction between the T cell receptor and the pMHC complex.

Most attention is focussed on the identification of small, that is, low molecular weight, compounds with therapeutic potential. This is generally because such compounds: are usually inexpensive to produce; can often be relatively easily and swiftly modified so as to provide variants of a “lead compound” which may have different properties; are often relatively stable, or can be modified to be stable, in the body, in particular compared to proteins and other biochemical substances; are less likely to provoke unwanted physiological reactions, like immune responses, than larger entities; and are more likely to be able to be administered orally because they are more likely to be able to pass the membrane barriers of the digestive tract into the blood, while less likely to be degraded by the digestive system.

Certain embodiments of the present invention relate to methods of screening libraries of candidate compounds for those which inhibit the TCR/pMHC complex interaction. A vast number of cellular interactions and cell responses are controlled by contacts made between cell surface receptors and soluble ligands, or ligands presented on the surfaces of other cells. These types of specific molecular contacts are of crucial importance to the correct biochemical regulation in the human body and are therefore being studied intensely. In many cases, the objective of such studies is to devise a means of modulating cellular responses in order to prevent or combat disease.

Recent advances in combinatorial chemistry, enabling relatively easy and cost-efficient production of very large compound libraries, has increased the scope for compound testing enormously. Now the limitations of screening programmes most often reside in the nature of the assays that can be employed and, in particular, how well these assays can be adapted to high throughput screening methods.

T cell receptors (TCR) have an off rate of approximately 0.05 s⁻¹ from an MHC/peptide complex. Such interactions have a K_{d} in the order of 10 μM. Interactions having K_{d}s above 10 mM tend to be non-specific. Because low affinity interactions having fast binding kinetics are so brief and weak, they are very difficult to detect.

The relatively weak (μM) affinities under investigation in the TCR/pMHC complex system place stringent requirements on the technology needed to screen for inhibition of the interaction. Many high throughput screen (HTS) assays involve homogenous methodologies, such as homogeneous time resolved fluorescence (HTRF). Such techniques have drawbacks when approaching low affinity interactions, as they require relatively high concentrations of the radioactive or fluorescent tracer molecules, typically resulting in low signal to background ratios.

Recently, Packard have developed a non-radioactive homogeneous assay technology specifically applicable to low affinity interactions—the AlphaScreen™ (Amplified Luminescent Proximity Homogenous Assay). In this assay, there are donor and acceptor beads, each with multiple copies of a protein pair bound to them. For example a TCR may be bound to the donor bead, and pMHC to the acceptor bead.

The Proteins may be attached by one of a number of different strategies e.g. biotin/streptavidin, or nickel chelate/6×HIS tag. The donor bead generates a chemical signal on laser excitation. The chemical signal is only detected by the acceptor bead if that bead is brought into close proximity by the interaction of the two proteins (Packard Instrument Company Technical Note AN001-ASc: Principles of AlphaScreen™. Amplified Luminescent Proximity Homogenous Assay.). The system has been used to detect low affinity interactions, for example the interaction between lectins and binding partners with K_{D} in the millimolar range (Homogeneous detection and measurement of micromolar affinity interactions using AlphaScreen. Chantal Illy et al. Abstract, SBS Conference 2001, Baltimore.)

The scintillation proximity assay (SPA) has also been used to screen compound libraries for inhibitors of the low affinity interaction between CD28 and B7 (K_{d} probably in the region of 4 μM (Van der Merwe et al (1997), Jenh et al,(1998)). SPA is a radioactive assay making use of beta particle emission from certain radioactive isotopes which transfers energy to a scintillant immobilised on the indicator surface.

The above screening techniques can be used to identify a suitable entity or, preferably, provide a set of candidate entities suitable for a further, secondary, screening step. The secondary screening preferably utilises a strategy for screening for compounds with the potential to interfere with sTCR/pMHC complex interactions based on Surface Plasmon Resonance (SPR), as described in WO01/22084.

As mentioned previously, the protein sequence may be associated with one or more diseases, such as an auto-immune disease, a cancer-associated disease, transplant rejection or graft-versus-host disease. The auto-immune disease may one or more of Hashimoto's disease, rheumatoid arthritis, osteo arthritis, dermatitis, chronic active hepatitis, pemphigus vulgaris, systemic lupus erythermatosus, myasthenia gravis, coeliac disease, Sjogren's syndrome, Addison's disease, insulin-dependant diabetes, Grave's disease, primary myxedema, Goodpasture's syndrome, tuberculoid leprosy, multiple sclerosis, ankylosing spondylitis, Reiter's disease, arthritis (post-salmonella, post-shigella, post-yersinia or post-gonococcal), uveitis, amyloidosis in rheumatoid arthritis, thyroiditis, psoriasis vulgaris, or idiopathic hemochromatosis.

The cancer-associated disease may be one or more of breast cancer, colon cancer, skin cancer, ovarian cancer, leukaemia, lymphoma, lung cancer, liver cancer, testicular cancer and nasophangeal malignancies.

A plurality of methods in accordance with the first aspect of the present invention can be carried out in parallel for a screen of a plurality of peptides in relation to a plurality of pMHCs. Thus, in a second aspect, the present invention provides a process which comprises two or more methods in accordance with the first aspect of the present invention, comprising two or more peptides as set out herein, and wherein each method comprises a different peptide in the pMHC.

The invention also provides an entity identifiable by the method of the first aspect or the process of the second aspect.

In a further aspect, the invention also provides the use of a pMHC complex, which complex binds to a T cell receptor, to identify a molecule which binds to the pMHC complex.

In many cases, the particular antigens involved in causing, for instance, autoimmune diseases, are not known. However, substantial information is available concerning the link between HLA type and disease. For example, significant HLA associations have been noted for renal, neurological, endocrine, gastrointestinal, respiratory, eye, dermatological, neurological and infectious diseases (Lechler et al., (2000)). An impressive body of data has been accumulated which links specific HLA antigens with particular disease states (this is summarised in Table 1). The relationships are influenced by linkage disequilibrium, a state where closely linked genes on a chromosome tend to remain associated rather than undergo genetic randomisation in a given population, so that the frequency of a pair of alleles occurring together is greater than the product of the individual gene frequencies. This could result from natural selection favouring a particular haplotype or from insufficient time elapsing since the first appearance of closely located alleles to allow to become randomly distributed throughout the population.

With the odd exception, such as idiopathic hemochromatosis and congenital adrenal hyperplasia resulting from a 21-hydroxylase deficiency, HLA-linked diseases are intimately bound up with immunological processes. The HLA-D related disorders are largely autoimmune with a tendency for DR3 to be associated with organ-specific diseases involving cell surface receptors. A popular model of MHC and disease association is that efficient binding of autoantigens by disease-associated MHC molecules leads to a T cell-mediated immune response and the resultant autoimmune sequelae. Alternative models have also been put forward; for example, Ridgway and Fathman (1998)) suggest that the association of MHC with autoimmunity results from “altered” thymic selection in which high-affinity self-reactive (potentially autoreactive) T cells escape negative selection.

TABLE 1
Association of HLA with disease

| | HLA | Relative |
|---|---|---|
| Disease | allele | risk |
| (a) Class II associated | | |
| Hashimoto's disease | DR5 | 3.2 |
| Rheumatoid arthritis | DR4 | 5.8 |
| Dermatitis herpetiformis | DR3 | 56.4 |
| Chronic active hepatitis (autoimmune) | DR3 | 13.9 |
| Pemphigus vulgaris | DR4 | 14 |
| Systemic lupus erythermatosus | DR3 | 6 |
| Myasthemia gravis | DR3 | 3 |
| Coeliac disease | DR3 | 10.8 |
| Sjogren's syndrome | DR3 | 9.7 |
| Addison's disease (adrenal) | DR3 | 6.3 |
| Insulin-dependent diabetes | DR3 | 5.0 |
| | DR4 | 6.8 |
| | DR3/4 | 14.3 |
| | DR2 | 0.2 |
| Thyrotoxicosis (Grave's) | DR3 | 3.7 |
| Primary myxedema | DR3 | 5.7 |
| Goodpasture's syndrome | DR2 | 13.1 |
| Tuberculoid leprosy | DR2 | 8.1 |
| Multiple sclerosis | DR2 | 4.8 |
| b) Class I, HLA-27 associated | | |
| Ankylosing spondylitis | B27 | 87.4 |
| Reiter's disease | B27 | 37.0 |
| Post-salmonella arthritis | B27 | 29.7 |
| Post-shigella arthritis | B27 | 20.7 |
| Post-yersinia arthritis | B27 | 17.6 |
| Post-gonococcal arthritis | B27 | 14.0 |
| Uveitis | B27 | 14.6 |
| Amyloidosis in rheumatoid arthritis | B27 | 8.2 |
| (c) Other Class I associations | | |
| Subacute thyroiditis | Bw35 | 13.7 |
| Psoriasis vulgaris | Cw6 | 13.3 |
| Idiopathic hemochromatosis | A3 | 8.2 |
| Myasthenia gravis | B8 | 4.4 |

(Data from Ryder et al. HLA and disease Registry 1979. Tissue Antigens, supplement 1979 & Marsh et al. The HLA FactsBook 2000. Pubs. Academic Press)

Class II Associations

A number of diseases have been linked to HLA Class II alleles, particularly DR2, DR3 and DR4. The most significant association appears to be that of dermatitis herpetiformis (coeliac disease of the skin), although associations have also been reported for coeliac disease itself, rheumatoid arthritis, insulin-dependent diabetes and multiple sclerosis. Other less common diseases with relatively high associations with HLA type are chronic active hepatitis, Sjogren's syndrome, Addison's disease and Goodpasture's syndrome.

The Genetic Contribution to the Pathogenesis of Rheumatoid Arthritis

Rheumatoid arthritis is a chronic inflammatory disease that primarily affects the joints and surrounding tissues. Although the cause of rheumatoid arthritis is unknown, infectious, genetic, and endocrine factors may play a role. The disease can occur at any age, but the peak incidence of disease onset is between the ages of 25 and 55. Women are affected 3 times more often than men and incidence increases with age. Approximately 3% of the population is affected. The onset of the disease is usually slow, with fatigue, loss of appetite, weakness, and vague muscular symptoms. Eventually, joint pain appears, with warmth, swelling, tenderness, and stiffness after inactivity of the joint. After having the disease for 10 to 15 years, about 20 percent of people will have had remission. Only 50% to 70% will remain capable of full-time employment and after 15 to 20 years, 10% of patients are invalids. The average life expectancy may be shortened by 3 to 7 years; factors contributing to death may be infection, gastrointestinal bleeding, and drug side effects. There is no known cure for rheumatoid arthritis and the disease usually requires life-long treatment. Current treatment includes various medications (including nonsteroidal anti-inflammatory drugs, gold compounds, immunosuppressive drugs), physical therapy, education, and possibly surgery aimed at relieving the signs and symptoms of the disease.

The association of HLA-DR4 or other HLA-DRB1 alleles encoding the shared (or rheumatoid) epitope has now been established in nearly every population. Similarly, the fact that the presence and gene dosage of HLA-DRB1 alleles affect the course and outcome of rheumatoid arthritis has likewise been seen in most (although not all) studies. Susceptibility to develop rheumatoid arthritis maps to a highly conserved amino acid motif expressed in the third hypervariable region of different HLA-DRB1 alleles. This motif, namely QKRAA, QRRAA or RRRAA helps the development of rheumatoid arthritis by an unknown mechanism. However, it has been established that the shared epitope can shape the T cell repertoire and interact with 70 kDa heat shock proteins (Reveille, Curr Opin Rheumatol 10(3):187-200 (1998)).

Coeliac Disease and Dermatitis Herpetiformis

Coeliac disease is one of the most common gastrointestinal disorders, affecting between 1:90 to 1:600 persons in Europe. The disease is a permanent intolerance to ingested gluten that results in immunologically mediated inflammatory damage to the small-intestinal mucosa. Coeliac disease is associated with HLA and non-HLA genes and with other immune disorders, notably juvenile diabetes and thyroid disease. The classic sprue syndrome of steatorrhea and malnutrition coupled with multiple deficiency states may be less common than more subtle and often monosymptomatic presentations of the disease. Diverse problems such as dental anomalies, short stature, osteopenic bone disease, lactose intolerance, infertility, and nonspecific abdominal pain among many others may be the only manifestations of coeliac disease. The treatment of coeliac disease is lifelong avoidance of dietary gluten.

Recent studies using human genome screening in families with multiple siblings suffering from coeliac disease have suggested the presence of at least four different chromosomes in the predisposition to suffer from coeliac disease. Other studies based on cytokine gene polymorphisms have found a strong association with a particular haplotype in the TNF locus; this haplotype carries a gene for a high secretor phenotype of TNFα. In addition to the strong association of coeliac disease with HLA-DR3, there is also evidence for an association with HLA-DQ. Both HLA-DQ2 and HLA-DQ8 restricted gliadin-specific T cells have been shown to produce IFNγ, which appears to be an indispensable cytokine in the damage to enterocytes encountered in the small intestine, since the histological changes can be blocked by anti-IFNγ antibodies in vitro (Pena (1998)).

Dermatitis herpetiformis (DH) is a pruritic, papulovesicular skin disease characterised in part by the presence of granular deposits of IgA at the dermal-epidermal junction, an associated gluten sensitive enteropathy, and a strong association with specific HLA types. Dermatitis herpetiformis is fairly uncommon, affecting around 1/10,000 persons in Europe and the US. Initial investigations revealed that 60% to 70% of patients with dermatitis herpetiformis expressed the HLA antigen B8 (normal subjects=21%). Further investigation of the HLA associations seen in patients with dermatitis herpetiformis has revealed an even higher frequency of the HLA class II antigens HLA-DR3 (DH=95%; normal=23%), HLA-DQw2 (DH=100%; normal=40%), and HLA-DPw1 (DH=42%; normal=11%) (Hall and Otley, (1991)). The association of the HLA-B8, HLA-DR3, HLA-DQw2 haplotype with Sjogren's syndrome, chronic hepatitis, Graves' disease, and other presumably immunologically mediated diseases, as well as the evidence that some normal HLA-B8, HLA-DR3 individuals have an abnormal in vitro lymphocyte response to wheat protein and mitogens and have abnormal Fc-IgG receptor-mediated functions, suggests that this HLA haplotype or genes linked closely to it may confer a generalized state of immune susceptibility on its carrier, the exact phenotypic expression of which depends on other genetic or environmental determinants.

Genetic Susceptibility Factors in Insulin-Dependent Diabetes Mellitus

Diabetes mellitus is a disease of metabolic dysfunction, most notably dysregulation of glucose metabolism, accompanied by characteristic long-term vascular and neurolgical complications. Diabetes has several clinical forms, each of which has a distinct etiology, clinical presentation and course. Insulin-dependent diabetes mellitus (type I diabetes; IDDM) is a relatively rare disease (compared with non-insulin-dependent diabetes mellitus, NIDDM), affecting one in 250 individuals in the US where there are approximately 10,000 to 15,000 new cases reported each year. The highest prevalence of IDDM is found in northern Europe, where more than 1 in every 150 Finns develop IDDM by the age of 15. In contrast, IDDM is less common in black and Asian populations where the frequency is less than half that among the white population.

IDDM is characterised by absolute insulin deficiency, making patients dependent on exogenous insulin for survival. Prior to the acute clinical onset of IDDM with symptoms of hyperglycemia there is a long asymptomatic preclinical period, during which insulin-producing beta cells are progressively destroyed. The autoimmune destruction of beta cells is associated with lymphocytic infiltration. In addition, abnormalities in the presentation of MHC Class I antigens on the cell surface have been identified in both animal models and in human diabetes. This immune abnormality may explain why humans become intolerant of self-antigens although it is not clear why only beta cells are preferentially destroyed.

The genetics of IDDM is complex, but a number of genes have been identified that are associated with the development of IDDM. Some HLA loci (in particular DR3 and DR4) are associated with an increased risk of developing IDDM, whereas other loci appear to be protective. Substitution of alanine, valine or serine for the more usual aspartic acid residue at position 57 of the β-chain encoded by the HLA-DQ locus has also been found to be closely associated with the increased risk of developing IDDM, although different combinations of DQA1 and DQB1 genes confer disease risk to differing degrees (Zamani and Cassiman, (1998)).

Genetics of Multiple Sclerosis

Multiple sclerosis (MS) is an inflammatory, demyelinating disease of the nervous system that is the most common cause of chronic neurological disability among young adults. MS is characterised by discrete demyelinating lesions throughout the CNS. The random nature of these lesions results in a wide variety of clinical features such as loss of sensations, muscle weakness, visual loss, cognitive impairment and fatigue. The mean age of onset is 30 years and females are more susceptible to MS than males by a factor that approaches 2:1. MS afflicts people almost worldwide, although there is epidemiologic variation in incidence and prevalence rates. The prevalence varies with latitude, affecting primarily northern Caucasian populations (e.g., 10 per 100,000 in southern USA, 300 per 100,000 in the Orkneys). Approximately 300,000 people are afflicted with MS in the US and 400,000 in Europe.

In North European populations, MS has been linked with Class I HLA alleles A3 and B7 and with Class II HLA alleles DR2, DQw1, DQA1 and DQB1. Particular HLA alleles (especially DR2) are considered to be risk factors for MS, and not simply genetic markers for the population of origin. However, this relationship is not universal and MS is linked to alleles other than DR2 in some populations (e.g., Jordanian Arabs and Japanese). This suggests that there is some heterogeneity in the contribution of HLA polymorphisms to MS susceptibility. Although particular alleles increase the risk for MS, no specific allele has yet been identified that is necessary for the development of MS. Overall, the contribution of the MHC to MS risk is believed to be fairly minor (Ebers and Dyment, (1998).

Class I Associations

The best known association of Class I HLA types with disease is that of HLA-B27 with anklyosing spondylitis and the related group of spondylarthropathies. Of the other Class I associations, the most important is probably that of HLA-Cw6 with psoriasis, although associations have also been reported for subacute thyroiditis, idiopathic hemochromatosis and myasthenia gravis.

HLA-B27 and the Seronegative Spondylarthropathies

The seronegative spondylarthropathies include ankylosing spondylitis, Reiter's syndrome and reactive arthritis, psoriatic arthritis, arthritis associated with ulcerative colitis and Crohn's disease, plus other forms which do not meet the criteria for definite categories and are called undifferentiated. Seronegative spondylarthropathies have common clinical and radiologic manifestations: inflammatory spinal pain, sacroiliitis, chest wall pain, peripheral arthritis, peripheral enthesitis, dactylitis, lesions of the lung apices, conjunctivitis, uveitis and aortic incompetence together with conduction disturbances.

In the 25 years since the initial reports of the association of HLA-B27 with ankylosing spondylitis and subsequently with Reiter's syndrome/reactive arthritis, psoriatic spondylitis, and the spondylitis of inflammatory bowel disease, the association of HLA-B27 with the seronegative spondyloarthropathies has remained one of the best examples of a disease association with a hereditary marker. The association of HLA-27 with in ankylosing spondylitis is quite remarkable, where up to 95% of patients are of B27 phenotype as compared to around 5% in controls. The prevalence of spondylarthropathies is directly correlated with the prevalence of the HLA-B27 antigen in the population. The highest prevalence of ankylosing spondylitis (4.5%) has been found in Canadian Haida Indians, where 50% of the population is B27 positive. Among Europeans, the frequency of the B27 antigen in the general population ranges from 3 to 13% and the prevalence of ankylosing spondylitis is estimated to be 0.1-0.23% (Olivieri et al. (1998)).

Experimental evidence from humans and transgenic rodents suggests that HLA-B27 itself may be involved in the pathogenesis of the spondyloarthropathies, and population and peptide-specificity analysis of HLA-B27 suggest it has a pathogenic function related to antigen presentation. In Reiter's syndrome (reactive arthritis) and ankylosing spondylitis putative roles for infectious agents have been proposed. However, the mechanism by which HLA-B27 and bacteria interact to cause arthritis is not clear and there are no clear correlations between peptide sequence, differential binding to B27 subtypes and recognition by peptide-specific T cell receptors (Lopez-Larrea et al. (1998)).

HLA-B27 and Uveitis

Uveitis involves inflammation of the uveal tract which includes the iris, ciliary body, and the choroid of the eye. Causes of uveitis can include allergy, infection, chemical exposure, trauma, or the cause may be unknown. The most common form of uveitis is anterior uveitis which affects the iris. The inflammation is associated with autoimmune diseases such as rheumatoid arthritis or ankylosing spondylitis. The disorder may affect only one eye and is most common in young and middle-aged people. Posterior uveitis affects the back portion of the uveal tract and may involve the choroid cell layer or the retinal cell layer or both. Inflammation causes spotty areas of scarring that correspond to areas with vision loss. The degree of vision loss depends on the amount and location of scarring.

In a recent study, Tay-Kearney et al Clinical features and associated systemic diseases of HLA-B27 uveitis. (Am J Ophthalmol 121(1):47-56 (1996)) reviewed the records of 148 patients with HLA-B27-associated uveitis. There were 127 (86%) white and 21 (14%) nonwhite patients, and a male-to-female ratio of 1.5:1. Acute anterior uveitis was noted in 129 patients (87%), and nonacute inflammation was noted in 19 (13%). An HLA-B27-associated systemic disorder was present in 83 patients (58%), 30 of whom were women, and it was diagnosed in 43 of the 83 patients as a result of the ophthalmologic consultation. Thirty-four (30%) of 112 patients had a family history of a spondyloarthropathy.

The Genetics of Psoriasis

Psoriasis is a disease characterised by uncontrolled proliferation of keratinocytes and recruitment of T cells into the skin. The disease affects approximately 1-2% of the Caucasian population and can occur in association with other inflammatory diseases such as Crohn's disease and in association with human immunodeficiency virus infection. Non-pustular psoriasis consists of two disease subtypes, type I and type II, which demonstrate distinct characteristics. Firstly the disease presents in different decades of life, in type I before the age of 40 years and later in type II Secondly, contrasting frequencies of HLA alleles are found: type I patients express predominantly MLA-Cw6, HLA-B57 and HLA-DR 7, whereas in type II patients HLA-Cw2 is over-represented. Finally, familial inheritance is found in type I but not in type II psoriasis. The study of concomitant diseases in psoriasis contributes to deciphering the distinct patterns of the disease. Defence against invading microorganisms seems better developed in psoriatics than in controls. This evolutionary benefit may have caused the overall high incidence of psoriasis of 2% (Henseler. (1998)).

Despite the HLA component, psoriasis in some families is inherited as an autosomal dominant trait with high penetrance. Susceptibility loci on other chromosomes have been identified following genome-wide linkage scans of large, multiply affected families although the extent of genetic heterogeneity and the role of environmental triggers and modifier genes is still not clear. The precise role of HLA also still needs to be defined. The isolation of novel susceptibility genes will provide insights into the precise biochemical pathways that control this disease. Such pathways will also reveal additional candidate genes that can be tested for molecular alterations resulting in disease susceptibility.

Thus, it can be seen that the association between certain HLA types and particular diseases has been well established. The best known of these is the association between the Class I molecule HLA-B27 and the spondylarthropathies, in particular ankylosing spondylitis. Despite the gene frequency of MA-B27 being relatively high in Caucasians (3-13%), this group of diseases is not common and the overall significance of the association is therefore somewhat reduced. Similarly, the HLA-DR3 allele (present in approximately 11% of the Caucasian population) is associated with a high risk (56.4) for the development of dermatitis herpetiformis, a relatively rare (1/10,000) skin disorder. However, there are associations between HLA types and more prevalent diseases with greater socioeconomic impact. For example, the relative risk of an individual with an HLA-DR4 allele developing rheumatoid arthritis is 5.8. Although this association is less than that between HLA-B27 and ankylosing spondylitis, rheumatoid arthritis affects approximately 3% of the population and the HLA-DR4 allele has a gene frequency of nearly 17% in Caucasian Americans. Similarly, although coelic disease has a relatively low risk associated with the presence of HLA-DR3 (10.8), this is a common haplotype and coelic disease is a prevalent gastrointestinal disorder.

In summary, there are a number of clinical diseases where there is an association with a particular HLA type (or types). The diseases with the most significant association with HLA type tend to be somewhat uncommon. However, there are a number of examples where the prevalence of the disease combined with the frequency of the HLA allele in the population make the association more significant, even if the risk associated with the particular HLA type is relatively low.

Genomics and proteomics techniques can be used to analyse the repertoire of genes and proteins expressed by distinct cell types. Studies comparing the expression profiles of cells in the diseased versus the healthy state can reveal proteins, or their respective genes, which, when overproduced, may produce an antigenic response causing—or adding to—the disease condition. Detection of T cell mediated responses against such antigens, and isolation and production of the components involved in triggering the response, can allow compounds with the potential to inhibit the responses and reverse the disease condition to be identified in screens.

Compounds that show increased binding to HLA complexes presenting peptides derived from a disease-related protein can be used as carrier molecules to direct effector drugs to the diseased cells. For cancer related antigens, this approach can enable the accumulation of effector drugs in the most relevant location. This allows for reduced systemic administration of the effector drug without loss of effect where the effect is most needed, i.e. within the cancerous tissue.

Preferred features of each aspect of the invention are as for each of the other aspects mutatis mutandis. The prior art documents mentioned herein are incorporated to the fullest extent permitted by law.

The invention will be described further with reference to the accompanying drawings in which:

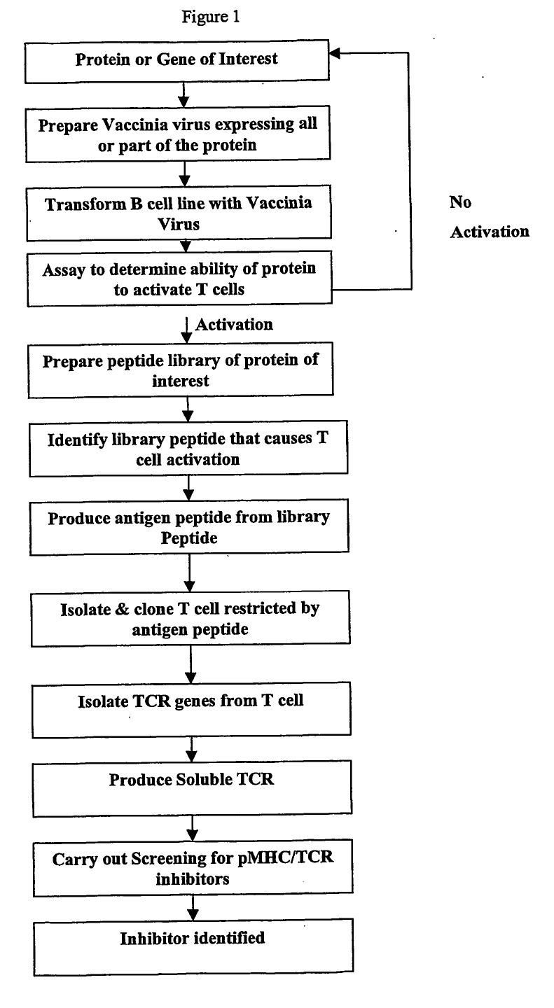

FIG. 1 is a schematic outline of a process for the identification of small compounds recognising putative antigenic protein peptides presented by HLA;

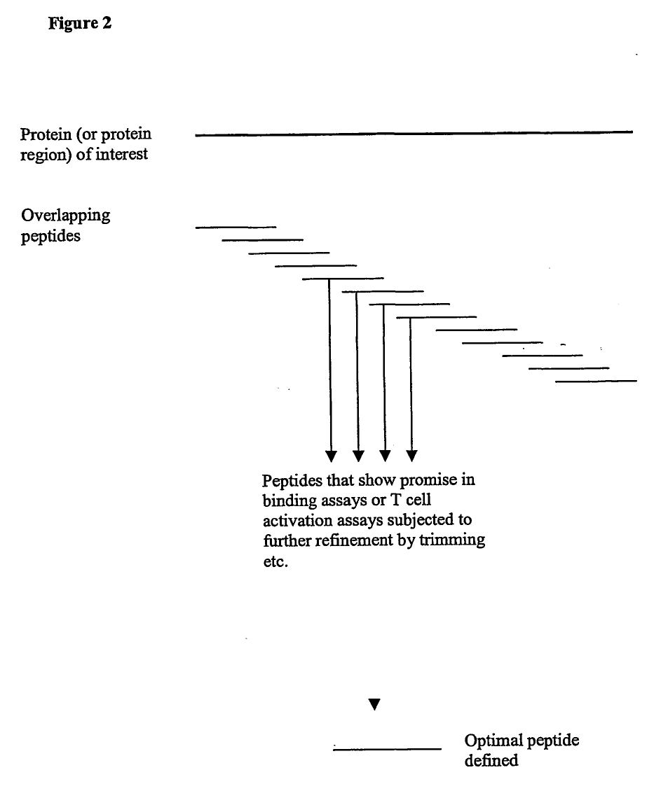

FIG. 2 is a schematic outline of a process for producing a peptide capable of complexing with a MHC

Class I Peptide Design

Peptides of between 8 and 11 amino acid residues long are synthesised with an overlap of one residue corresponding to either the full length of the protein or preferably a region of the protein identified by truncation analysis (see example 1). The overlap has to be one residue because of the binding constraints of the peptide groove in class I MHC (Rammensee et al. 1993).

Class II Peptide Design

Peptides of between 12 and 30 amino acid residues long are synthesised with an overlap of between 5 and 10 residues corresponding to either the full length of the protein or preferably a region of the protein identified by truncation analysis (see example 1). The peptide binding groove of class II MHC is not as constrained as in class I and so the ends of the peptide can overhang the ends of the groove (Germain, 1994).

In addition to conventional peptides, labels such as biotin or fluorochromes can be incorporated to allow for the detection of bound peptides on antigen presenting cells in FACScan experiments;

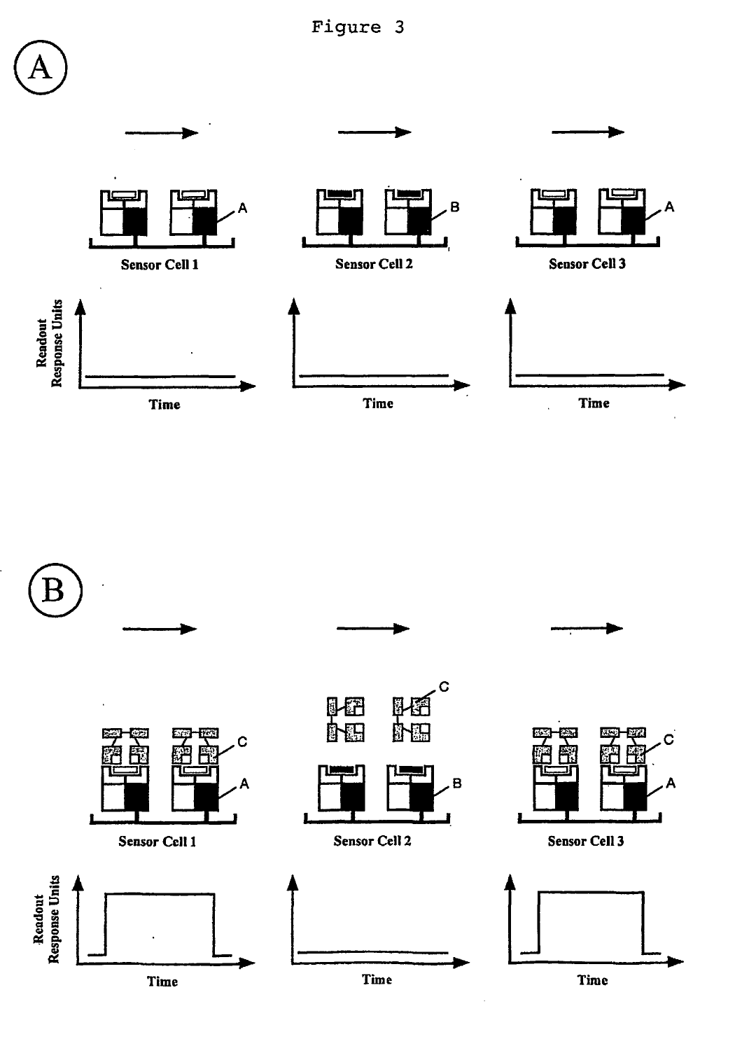

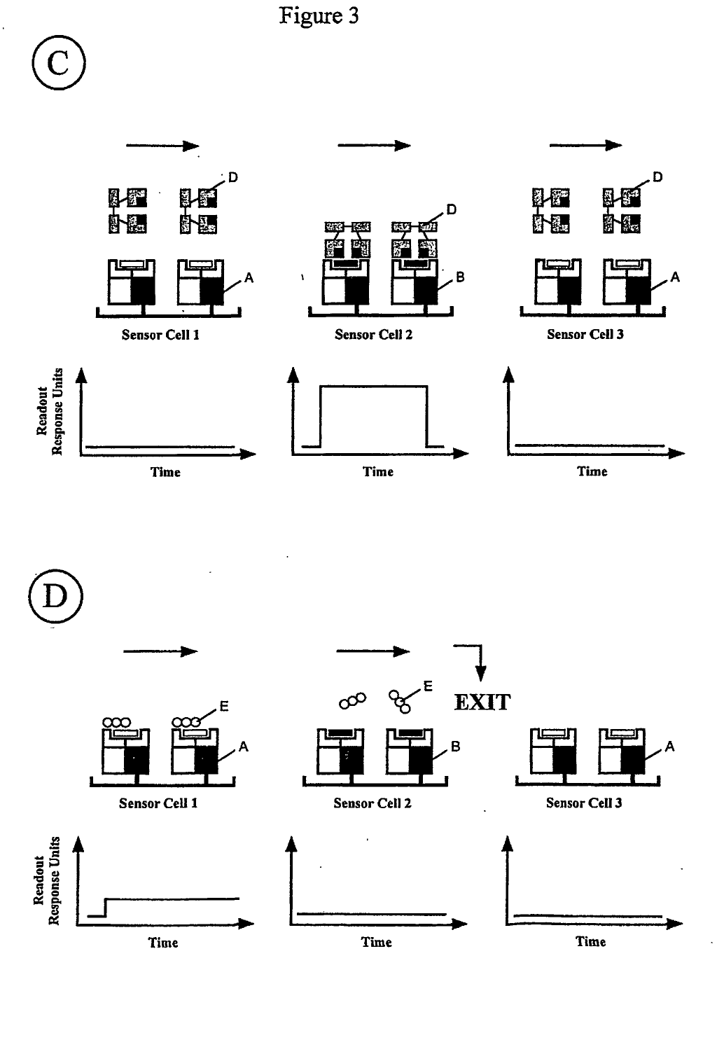

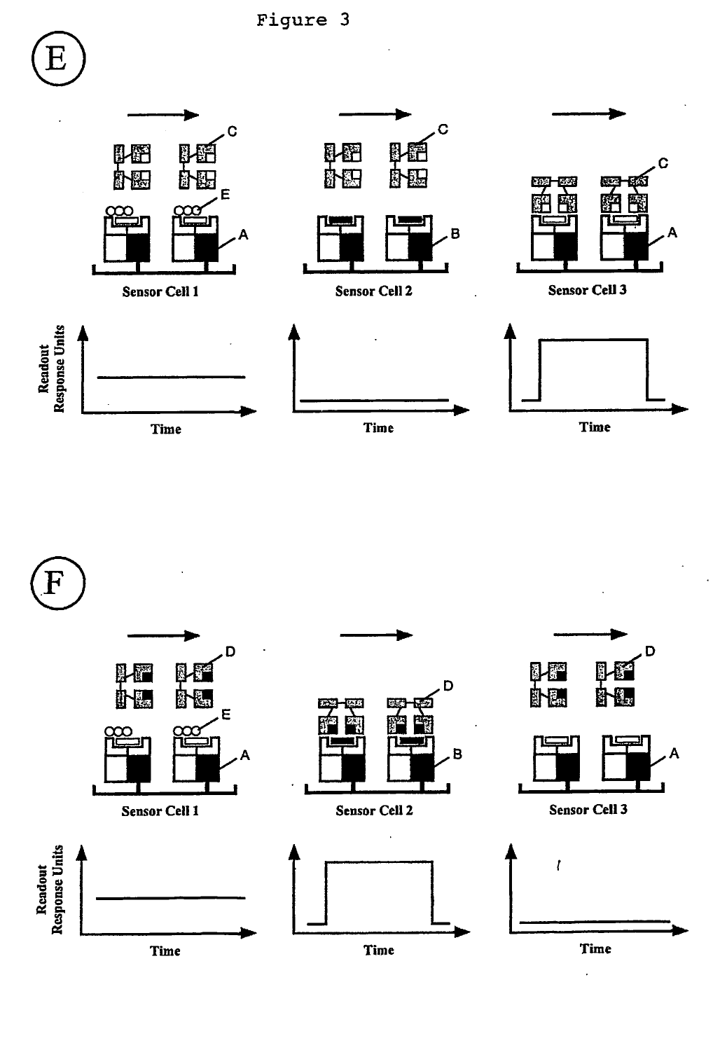

FIG. 3 is a schematic representation of the steps in a method in accordance with the present invention for screening for an inhibitor which inhibits the binding of a T cell receptor to an MHC molecule complexed with a specific peptide antigen.

The invention is now described with reference to the following non-limiting Examples.

### EXAMPLES

### Example 1

### Constructing Recombinant Vaccinia Virus (rVV) Expressing the Protein of Interest

To allow the testing, in cell culture, of the immune response elicited by epitopes of the protein of interest, rVV expressing the protein or protein fragment of interest is constructed using techniques as described by Broder and Earl (1999).

The gene of interest, or gene fragments of interest (truncation analysis), is amplified from cDNA, using the polymerase chain reaction (PCR), using synthetic DNA primers. The amplified cDNA is inserted into a transfer vector such as pSC 11 (Chakrabarti S et al, 1985) or pTKgptF1s (Falkrer F G and Moss B, 1988).

Incorporation of the gene, or gene fragment, of interest into the viral genome is by homologous recombination between the Vaccinia virus (e.g Wild type strain WR; American tissue culture collection (ATCC), Manassas, Va., USA; ATCC # VR1354) DNA and the recombinant transfer vector. Protocols for generating the rW are described in detail in Broder Earl, (1999) and Mazzara et al, (1993).

### Example 2

### Generating Epstein Barr Virus (EBV)-Transformed Human B Cell Lines

B-cell transformation by EBV is based on standard methods (Rickinson et al, 1984; Tasato et al, 1997). EBV is produced from EBV-transformed marmoset producer cell line B95-8 cells (American Tissue Cell Collection, Manassas, Va., (ATCC) Catalogue number CRL-1612) (Luka et al, 1978). Supernatants are collected after 3 days from B95-8 cells at 10⁶ cells/ml in supplemented RPMI-1640 medium. Supernatant is passed through a 0.45 μm sterile filter and stored at −80 C. Fresh purified blood mononuclear cells (10⁷ cell in 2.5 ml) is mixed with 2.5 ml of thawed EBV stock. Transformation may be inhibited by outgrowth of EBV-specific cytotoxic T lymphocytes, therefore 0.5 μg/ml cyclosporin A is added to cultures. Cells are maintained for 3 weeks at 37° C. and transformed B lymphoblasts are amplified by regular passage.

rVV expressing the protein of interest (Example 1) are transfected into the EBV transformed B cell lines using Lipofectin (Life Sciences). Cells transfected with Vaccinia Virus without the gene insert (non-rVV) are used in negative control experiments

### Example 3

### HLA Matched Continuous Cell Lines

An alternative to the approach taken in Example 2 involves using continuous cell lines. (sources such as ATCC) expressing the patients HLA type. rVV expressing the protein of interest (Example 1) is transfected into the HLA matched cell lines using Lipofectin (Life Sciences).

### Example 4

### Cytokine-Based T Cell Activation Assay Using Vaccinia Transfected Cells for Identifying T Cell Activation

Blood samples are taken from two subjects:

Subject A is a HLA typed patient suffering from the disease of interest

Subject B is a healthy patient, HLA matched with Subject A

Peripheral Blood Lymphocytes (PBL's) are prepared from both blood samples.

The vaccinia transfected cell lines (from Examples 2 & 3) are used as targets in a cytokine assay. The target cells are placed into microtitre plates with PBL's prepared from the blood of Subject A or Subject B. Supernatants are harvested after 2-16 hours.

Experimental Design

| | |
|---|---|
| Test Samples - | rVV transfected cells co-cultured with PBL from |
| | Subject A or B |
| Control - | non-rVV transfected cells co-cultured with PBL from |
| | Subject A or B |

A standard cytokine assay, for example a macrophage inflammatory protein-1β (MIP-1β) assay (Quantikine®)-Human MIP-1β Immunoassay, Cat No: DMB00, R&D Systems Europe, Abingdon UK) is carried out on the supernatant in accordance with the manufacturers instructions.

Alternative assays based on the cytokines IFN-γ and RANTES may also be used. Chemokines are cell activation makers expressed by a range of cells including CTL. Therefore, any increase in cytokine production in the Test samples compared with the controls indicates the presence of a T cell restricted by a pMHC presenting a portion of the protein expressed by the vaccinia

### Example 5

### Identiying Reactive T Cells From Alternative Sources.

K562 cells (Lozzio and Lozzio (1975)) that do not express endogenous class I HLA genes (Rosa, and Fellous (1982)) are transfected to express a particular HLA gene. Further transfection of this cell line with a plasmid expressing the protein under investigation generates a double transfected cell line that presents peptides derived from the protein of interest by the particular HLA molecule. The double transfected cell line is used to probe blood samples from HLA matched or mismatched donors for T cell reactivity. The single transfected cell line is used as a control to evaluate reactivity against endogenous K562 proteins presented by the HLA. Increased T cell reactivity against the double transfected cell line measured by EliSpot or equivalent assay compared to the response against the HLA single transfected K562 cells strongly indicates the presence in the blood sample of T cells reactive against peptides deriving from the protein and presented by the particular HLA.

### Example 6

### Expanding MHC Class H Restricted T Cell Clones

For expansion of antigen-specific class II-restricted T cells, CD4⁺ T cells are purified from a patient's PBMCs using antibody coated magnetic beads (Minimacs, Miltenyi Biotech), and are cultured with EBV-transformed autologous mononuclear cells, together with recombinant protein and IL-2. Recombinant proteins are expressed in E. coli as inclusion bodies or as secreted fusion proteins and are purified prior to use in T cell stimulation assays. Lymphoblasts from bulk cultures are cloned by limiting dilution using antigen presenting cells (APCs), recombinant protein and IL-2. Resulting clones are expanded by culturing with PHA, allogeneic irradiated PBMCs and IL-2. Antigenic specificityis verified by testing proliferative response of each T cell clone to intact protein presented by autologous MHCs. The restriction element of each clone is determined using MHC-matched APC's.

### Example 7

### Identifying Antigen Epitopes by Peptide Mapping

Following identification of a target protein using Examples 1-6, the protein is further analysed by peptide mapping. Antigens recognized by cytotoxic T cells are short peptides (8-11 amino acids) bound to class I MHC molecules. These peptides are produced by the degradation of proteins that are produced inside the cell (Rammensee et al. 1993). Class II MHC molecules are known to present peptides derived from exogenous proteins that are taken up and processed intracellularly and transported to the cell surface where the complex is anchored in the membrane. The peptides presented by Class II HLA are 12-24 amino acids in length (Germain, 1994). There are a number of companies and institutions that provide peptide synthesis services. For example the Institute of Animal Health (Berkshire, UK).

FIG. 2 shows the overall concept of peptide mapping for class I and class II systems. Overlapping peptides derived from the protein sequence of interest are tested using an appropriate T cell assay (see Example 8). For antigens presented by MHC Class I, peptides of between 8 and 11 amino acid residues long, corresponding to either the full length of the protein or preferably a region of the protein identified by truncation analysis, are synthesised with an overlap of one residue. The overlap has to be one residue due to the binding constraints of the peptide groove in class I MHC (Rammensee et al. 1993). For antigens presented by MHC Class II, peptides of between 12 and 30 amino acid residues long, corresponding to either the full length of the protein or preferably a region of the protein identified by truncation analysis (see example 6), are synthesised with an overlap of between 5 and 10 residues. The peptide-binding groove of class II MHC is not as constrained as in class I therefore the ends of the peptide can overhang the ends of the groove (Germain, 1994). A detailed knowledge on the peptide epitope at the level of single amino acids is defineable in this manner (see Engelhard V H, 1994, Masucci M G, 1993, Banks T A, et al, 1993, Nayersina R et al, 1993, Khanna R et al, 1993, Feltkamp M C W et al, 1993).

### Example 8

### Diseases Where Protein Polymorphisms are Thought to be Involved

Antigens of this category correspond to peptides derived from regions of proteins that are mutated in tumour cells. In this case, the search for peptide epitopes is narrowed down to that region in which the mutation occurs. Listed below are a number of examples of such proteins:

Houbiers et al, (1993) examined peptides encoded by the wild-type p53 gene and by each of 32 published mutant forms. Their ability to bind to HLA-A2 was assessed using a computer scoring system, followed by binding assays. They successfully generated peptide-specific CD8+CTL clones to the mutant proteins. Similarly, Yanuck et al, (1993) examined a point mutation found in p53 gene product from a human lung carcinoma. Balb/c mice were immunized with spleen cells pulsed with peptide corresponding to the 21 amino-acid sequence encompassing a point mutation in the mutant p53 gene product from the human lung cancer. The mutation created a new Kd class I molecule binding motif. CTLs killed Balb/c fibroblasts transfected with the mutant gene. This demonstrates that CTLs can be generated by peptide immunization and can lyse tumour cells expressing that mutation. Similar systems have been explored by the following: Fossum et al, (1994), Skipper and Stauss, (1993).

### Example 9

### T Cell Assays

A functional screen is employed to identify antigenic epitopes capable of eliciting T cell responses to proteins differentially expressed in disease states.

Primary Bulk Culture

Antigen-specific T cells are obtained by culturing patient peripheral blood mononuclear cells (PBMCs) with Epstein Barr Virus (EBV)-transformed autologous mononuclear cells transfected with recombinant vaccinia virus expressing the protein of interest (see Examples 1-3). Prior to culture, CD4⁺ and CD8⁺ T cell subsets are separated using antibody coated magnetic beads (Minimacs, Miltenyi Biotech). The resulting lymphoblasts from CD4+ or CD8+bulk cultures are cloned by limiting dilution using transfected autologous PBMCs and IL-2. Resulting clones are expanded by culturing with PHA, allogeneic irradiated PBMCs and IL-2. Antigenic specificity is verified by testing proliferative response of each T cell clone to transfected autologous MHCs. The restriction element of each clone is determined using MHC-matched APC's.

T Cell Proliferation Assay

CD4⁺ T cell clones are stimulated by co-culture for 72 hr with 5×10⁴ autologous irradiated EBV-transformed PBMCs transfected with recombinant vaccinia virus expressing the protein, or pulsed with 5 ug/ml peptide (see below). After 54 hr, ³H-thymidine is added and incorporation is measured after a further 18 hr culture.

Cytotoxicity

CD8⁺ T cells are primed in vitro by culturing with peptide and autologous irradiated APCs in the presence of IL-2 and IL-7, prior to culturing them with peptide pulsed 51Cr-labeled target cells as described (Jager et al. 2000). Non-peptide puilsed target cells are used as controls for non-specific cytotoxicity.

ELISPOT Analysis

Analysis of antigen-reactive T cells from patients and T cell lines produced by antigen-driven proliferation in vitro is investigated by ELISPOT analysis using cytokine specific monoclonal antibodies. Of particular interest is the cytokines associated with Th1 or cellular immunity eg. TNF-α, γ-IFN,GM-CSF, given that CD8+ T cells are capable of producing Th1 cytokines in a class I-restricted and tumor-specific fashion, and are therefore potentially capable of exerting a localized positive, noncytolytic effect on cellular immunity (Chakraborty & Mukherji, 1998). Standard ELISPOT protocols are used (Current Protocols in Immunology, John Wiley and S, Inc, Chapter 6), although in some instances Sf9 insect cells expressing class I or class II molecules are used as APCs in order to reduce background signal (Janetzki et al., 2000)

### Example 10

### Peptide Design

Peptides from the protein are synthesized as described in Example 7, and are tested for their ability to stimulate T cell clones. In some instances, knowledge of the restricting MHC haploype permits rational choice of peptide sequence, based on paradigms set for specific MHC anchor residues. The predictive programs employed include programmes available at the following web sites: (http://syfpeithi.bmi-heidelberg.com/) and (http://sdmc.krdl.org.sg:8080/fimm/). For instances where the restricting MHC allele does not allow for this type of predictive analysis (e.g. for many MHC class II alleles), a redundant strategy is taken in which all possible peptides are synthesized in overlapping fashion (see Example 7).

### Example 11

### Altered Peptide Ligands

T cell responses are governed by both the affinity of a peptide for the presenting MHC molecule, and the affinity of the peptide-MHC (pMHC) for the T cell receptor (TCR). A current limitation of cancer immunotherapy is that host tumour-associated antigens elicit only moderately weak T cell responses, presumably due to selection of a self-tolerant T cell repertoire. There are several reports describing how weakly stimulating peptides (antagonists) have been converted into strongly stimulating peptides (agonists) simply by altering key MHC-peptide anchor residues or TCR contact residues (Lyons et al., 1996; Alam et al 1999; Baker et al., 2000). Significantly, it has recently been shown that peptide substitutions in an immunogen designed to increase the stability of the pMHC-TCR complex, result in enhanced activation and expansion of T cells in vivo (Slansky et al, 2000).

Following identification of the key antigenic peptide(s) from the protein of interest, knowledge of the restricting MHC allele may be used to identify candidate MHC anchor residues in the peptide that may be mutated to facilitate enhanced binding. T cell stimulation assays are carried out using titrated amounts of peptide variants in dose-response experiments to screen for enhanced T cell stimulation. For CD8+class I restricted T cell clones, cytolytic activity is assessed using MHC-matched peptide pulsed target cells (as described above), whereas CD4⁺ class II restricted T cell stimulation is measured by IL-2 release from stimulation cultures using cytokine-dependent indicator cell lines (Current Protocols in Immunology, John Wiley and S, Inc, Chapter 7).

It is anticipated that selection of peptide variants that impart stability to the pMHC-TCR complex will identify T cell clones with superior antigenic activity.

### Example 12

### Generating T-Cell Clones

HLA-peptide tetrameric complexes are used to isolate antigen specific T-cell clones. HLA-peptide tetrameric complex production is described in WO 96/26962.

Specific T-cell clones may also be isolated using limiting dilution techniques as previously described (Carmichael 2000).

Soluble peptide-MHC tetramers are produced using a similar method to that described by Altman et al, 1996. Recombinant MHC class I heavy chain and β2 microglobulin, or α and β chains of MHC class II are produced in Escherichia coli cells transformed with the relevant expression plasmid. HLA complexes are folded using 30 mg of heavy chain protein, 25 mg of β2 microglobulin (when applicable) and 10 mg of synthetic protein.

The MHC complexes are biotinylated using 5 μg/ml purified BirA enzymes, 0.5 mM biotin and 5 mM ATP. The reaction is incubated at room temperature for 16 h. MHC-peptide complexes are recovered by FPLC purification and ion exchange chromatography. Tetramers are made by mixing biotinylated complexes with streptavidin-PE (Sigma Chemicals Co) at a molar ratio of 4:1. The labelled tetramers are concentrated to 3-4 mg/ml and stored in PBS at 4° C.

Peripheral blood mononuclear cells (PBMC) are isolated from venous blood from patients using Lymphoprep centrifuge gradient. PBMC (1×10⁶ cells) are incubated at 4° C. for 30 minutes in 2 μl of a solution of phycoerythrin labelled tetrameric complexes at a concentration of HLA molecules of 0.5 mg/ml in PBS with 0.1% bovine serum albumin. The cells are washed and incubated at 4° C. for a further 30 minutes with saturating concentration of anti-CD8 monoclonal antibody. After washing, the samples are sorted in a Becton Dickinson FACSVantage using CELLQuest software. Small lymphocytes are gated for forward and side scatter profile, before cloning according to tetramer/CD8 double staining.

Single cells are sorted directly into 96-well plates previously coated with anti-CD3 and anti-CD28 both at 100 ng/ml in PBS. In addition each well contains with irradiator feeder cells 10⁵ EBV lymphoblastic cells lines and autologus peripheral blood lymphocytes 106. Cloning plates are incubated at 37° C. in 5% CO₂ for 10-14 days. The proliferating clones are expanded in RPMI 1640 medium, followed by stimulating with 5 μg/ml PHA with irradiated allogenic 10⁶ PBMC and 10⁵ EBV transformed lymphoblastic cell line.

### Example 13

### Molecular Cloning of TCR Genes from T cell Clones.

T cell receptor genes (TCRs) are isolated from T cell clones by combined reverse transcription of total RNA and specific PCR amplification of the relevant genes.

A suitable number of T cells, typically 1-5 million, are lysed in a suitable lysis buffer. A number of systems are commercially available like the “mRNA Capture Kit” from Boehringer Mannheim or the “TRI reagent” system from Sigma. RNA is isolated according to the manufacturers recommendations. The cDNA synthesis reaction is primed either by oligo-dT nucleotides 12-18 residues long or by oligo nucleotides specific for a region in the TCR constant domain. A number of companies provide suitable reverse transcriptase enzyme for cDNA synthesis, for example, Stratagene's AMV reverse transcriptase or Qiagen's Omniscript. The exact cDNA synthesis protocol is not critical although the use of a highly processive enzyme is recommended in order to optimise full-length synthesis.

Should the subsequent PCR amplification strategy use poly-C forward primers on G-tailed cDNA as described by Moss et al. (1991) or be based on ligating adaptor molecules to double stranded cDNA as described Chen et al. (1992) a size exclusion purification step for isolation of full length cDNA is recommended. A number of techniques may be employed for size fractionation of cDNA. cDNA can be separated on sequencing gels and the desired fraction eluted from the gel for further processing by PCR. Alternatively, the cDNA may be size fractionated using spin column chromatography; “Chroma-spin-400” or “Chroma-spin-1000” columns from Clontech are suitable for isolation of high molecular weight cDNA.

PCR reactions are performed using a poly-C anchor primer containing a suitable restriction enzyme recognition sequence and an alpha- or beta-chain specific primer annealing in the respective TCR constant region. Another suitable restriction enzyme recognition sequence is added to the constant domain specific primer to facilitate subsequent molecular cloning. PCR is performed using Pfu polymerase or another DNA polymerase with proof-reading activity. Taq- or similar DNA polymerase can be used but is more prone to generate errors due to an intrinsically lower fidelity.

PCR fragments are digested with appropriate restriction enzymes and ligated into recipient plasmid vectors prepared with the relevant restriction enzymes by standard techniques (Sambrook and Russell, 2001). The sequence of the plasmids is verified by manual or automated DNA sequencing.

### Example 14

### Producing and Characterising Soluble TCR

A recombinant soluble form of the heterodimeric TCR molecule is engineered. Each chain consists of membrane-distal and —proximal immunoglobulin domains which are fused via a short flexible linker to a coiled coil motif which helps stabilise the heterodimer.

The TCR constant domains are truncated immediately before cysteine residues which, in vivo, form an interchain disulphide bond. Consequently, the two chains pair by non-covalent quatemary contacts. As the Fos-Jun zipper peptide heterodimers are also capable of forming an interchain disulphide immediately N-terminal to the linker used (O'Shea et al 1989), the alignment of the two chains relative to each other is predicted to be optimal. Fusion proteins may be joined in a manner which is compatible with each of the separate components, in order to avoid disturbing either structure.

cDNA encoding alpha and beta chains of a TCR is obtained from a CTL clone (JM22) by anchored PCR as described previously (Moss et al 1991).

Alpha and beta TCR-zipper constructs pTCRα-Jun and pTCRβ-Fos are separately constructed by amplifying the variable and constant domain of each chain using standard PCR technology and splicing products onto leucine zipper domains from the eukaryotic transcription factors Jun and Fos respectively. These 40 amino acid long sequences have been shown to specifically heterodimerise when refolded from synthetic peptides, without the need for a covalent interchain linkage (O'Shea et al 1989).

Primers are designed to incorporate a high AT content immediately 3′ to the initiation codon (to destabilise mRNA secondary structure) and using E. coli codon preferences, in order to maximise expression (Gao et al. (1998)).

The spare cysteine in the TCR beta constant domain is mutated to serine to prevent incorrect disulphide bonding during refolding.

DNA constructs are ligated separately into the E. coli expression vector pGMT7. Plasmid digests and DNA sequencing are used to confirm that the constructs are correct.

Expression of TCR zipper chains and purification of denatured inclusion bodies: The pGMT7 expression plasmids pTCRα-Jun and pTCRβ-Fos respectively are transformed separately into E. coli strain BL21 (DE3) pLysS, and single ampicillin-resistant colonies are grown at 37° C. in TYP (ampicillin 100 μg/ml) medium to OD₆₀₀ of 0.4 before inducing protein expression with 0.5 mM IPTG. Cells are harvested three hours post-induction by centrifugation for 30 minutes at 400 rpm in a Beckman J-6B. Cell pellets are resuspended in a buffer containing 50 mM Tris-HCl, 25% (w/v) sucrose, 1 mM NaEDTA, 0.1% (w/v) NaAzide, 10 mM DTT, pH 8.0. After an overnight freeze-thaw step, resuspended cells are sonicated in 1 minute bursts for a total of around 10 minutes in a Milsonix XL2020 sonicator using a standard 12 mm diameter probe. Inclusion body pellets are recovered by centrifugation for 30 minutes at 13000 rpm in a Beckman J2-21 centrifuge. Three detergent washes are then carried out to remove cell debris and membrane components. Each time the inclusion body pellet is homogenised in a Triton buffer (50 mM Tris-HCL 0.5% Triton-X100, 200 mM NaCI, 10 mM NaEDTA, 0.1% (w/v) NaAzide, 2 mM DTT, pH 8.0) before being pelleted by centrifugation for 15 minutes at 13000 rpm in a Beckman J2-21. Detergent and salt is then removed by a similar wash in the following buffer: 50 mM Tris-HCl, 1 mM NaEDTA, 0.1% (w/v) NaAzide, 2 mM DTT, pH 8.0. Finally, the TCRα-Jun and TCRβ-Fos inclusion body pellets are dissolved separately in a urea solution (50 mM MES, 8M urea, 10 mM NaEDTA, 2 mM DTT, pH 6.5) for 3 to 4 hours at 4° C. Insoluble material is pelleted by centrifugation for 30 minutes at 13000 rpm in a Beckman J2-21, and the supernatant is divided into 1 ml aliquots and frozen at −70° C. Inclusion bodies solubilised in urea are quantitated with a Bradford dye-binding assay (Biorad). For each chain a yield of around 100 mg of purified inclusion body is obtained from one litre of culture. Each inclusion body (TCRα-Jun, TCRβ-Fos) is solubilised in urea solution at a concentration of around 20 mg/ml, and is estimated from gel analysis to be around 90% pure in this form (data not shown).

Co-Refolding of TCR-Zipper Fusion Proteins:

Initial refolding experiments using a standard refolding buffer (100 mM Tris pH 8.5, 1M L-Arginine, 2 mM EDTA, 5 mM reduced Glutathione, 0.5 mM oxidised Glutathione, 0.2 mM PMSF) results in severe protein precipitation which is dependent upon the presence of the zipper domains. The fact that this phenomenon occurs at concentrations below the dissociation constant of zipper dimerisation (i.e. when most zipper helices are expected to be monomeric) suggests that additional forces are stabilising misfolded species. The most likely explanation is that the entirely alpha-helical zipper domains fold first and that their transient heterodimerisation induces inter-chain aggregation of partially folded intermediates of the more complex immunoglobulin domains. The refolding buffer is therefore altered to include 5M urea in order to prevent hydrophobic interactions between partially folded immunoglobulin domains and allow individual chains to fold completely before heterodimerisation. This step is sufficient to prevent precipitation occurring, and allows correctly folded TCR-zipper heterodimers to assemble with acceptable yields using the following protocol.

Urea-solubilised stocks of TCR-zipper chains TCRα-Jun and TCRβ-Fos are renatured by dilution co-refolding. Approximately 30 mg (i.e. 1 μMole) of each solubilised inclusion body chain is thawed from frozen stocks and a further pulse of DTT (4 μmoles/ml) is added to ensure complete reduction of cysteine residues. Samples are then mixed and the mixture diluted into 15 ml of a guanidine solution (6 M Guanidine-hydrochloride, 10 mM Sodium Acetate, 10 mM EDTA), to ensure complete chain denaturation. The guanidine solution containing fully reduced and denatured TCR-zipper chains is then injected into 1 litre of the following refolding buffer: 100 mM Tris pH 8.5, 400 mM L-Arginine, 2 mM EDTA, 5 mM reduced Glutathione, 0.5 mM oxidised Glutathione, 5M urea, 0.2 mM PMSF. The solution is left for 24 hrs. The refold is then dialysed twice, firstly against 10 litres of 100 mM urea, secondly against 10 litres of 100 mM urea, 10 mM Tris pH 8.0. Both refolding and dialysis steps are carried out at 2-8° C.

Purifying Refolded TCR-Zipper:

TCR-zipper TCRzip is separated from degradation products and impurities by loading the dialysed refold onto a POROS 10HQ analytical anion exchange column in seven 200 ml aliquots and eluting bound protein with a gradient of 0-400 mM NaCI over 50 column volumes using a BioCad workstation (Perseptive Biosystems). Non-covalently associated heterodimer elutes in a single peak at approximately 100 mM NaCI. Peak fractions (typically containing heterodimer at a concentration of 100-300 μg/ml) are stored at 4° C. before being pooled and concentrated. The yield of heterodimer is approximately 15%.

Characterising the Refolded TCR-Zipper TCRzip:

The TCRzip heterodimer purified by anion exchange elutes as an approximately 70 kDa protein from a Superdex 200 gel filtration sizing column (Pharmacia). It is important to include gel filtration steps prior to surface plasmon resonance binding analysis since accurate affinity and kinetic measurements rely on monomeric interactions taking place. In this way, higher order aggregates can be excluded from the soluble protein fraction used for analysis. In particular, aggregates cause artifactually slow association and dissociation rate constants to be detected.

The oxidation state of each chain is examined by a reducing/non-reducing gel analysis. In the presence of SDS, the non-covalently associated heterodimer is dissociated into alpha and beta chains. If DTT is used in loading buffer, the two chains run either side of the 31 kDa marker. In the absence of such denaturants both chains still behave as a single species, but the mobility of each increases, which suggests each chain has formed a single, disulphide-bonded species (Garboczi et al 1996).

Specific Binding of Refolded TCR-Zipper to Peptide-MHC Complexes:

A surface plasmon resonance biosensor (BIAcore) is used to analyse the binding of a TCR-zipper to its peptide-MHC ligand (see FIG. 3). This is facilitated by producing single pMHC complexes (described below) which can be immobilised to a streptavidin-coated binding surface in a semi-oriented fashion, allowing efficient testing of the binding of a soluble T-cell receptor to up to four different pMHCs (immobilised on separate flow cells) simultaneously. Manual injection of HLA complex allows the precise level of immobilised class I molecules to be manipulated easily.

Such immobilised complexes are capable of binding T-cell receptors and the coreceptor CD8αα, both of which may be injected in the soluble phase. Specific binding of TCR-zipper is obtained even at low concentrations (at least 40 μg/ml), implying the TCR zipper is relatively stable. The pMHC binding properties of sTCR are qualitatively and quantitatively similar if sTCR is used either in the soluble or immobilised phase. This is a control for partial activity of soluble species and may also suggest that biotinylated pMHC complexes are biologically as active as non-biotinylated complexes.

Preparing Chemically Biotinylated HLA Complexes:

Methods for producing soluble, recombinant single peptide class I HLA complexes have already been described (Garboczi et al 1992). These are modified in order to produce HLA complexes which have β-2-microglobulin domains chemically biotinylated and may therefore be immobilised to a streptavidin coated binding chip and used for surface plasmon binding studies.

β-2-microglobulin is expressed and 40 mg refolded in a standard refolding buffer (100 mM Tris pH 8.0, 400 mM L-Arginine, 2 mM EDTA, 5 mM reduced Glutathione, 0.5 mM oxidised Glutathione, 0.1 mM PMSF) essentially as described (Garboczi et al 1992). After an optional gel filtration step, protein is exchanged to 0.1M Sodium Borate pH 8.8, and finally concentrated to 5-10 mg/ml. β-2-microglobulin is also quantitated using the Bradford assay (Biorad). A 5 molar excess of biotin hydroxysuccinimide (Sigma) is added from a stock made up at 10 mg/ml in DMSO. The reaction is left for 1 hour at room temperature, and is stopped with 20 μl of 1M Ammonium Chloride/250 μg of biotin ester. Refolded HLA complex is separated from free biotin and free biotinylated beta-2-microglobulin using a Superdex 200 gel filtration sizing column (Pharmacia). Streptavidin is immobilised by standard amine coupling methods.

Alternatively, proteins may be engineered to contain a specific biotinylation sequence which is recognised by the bacterial enzyme, BirA (Barker & Campbell, (1981 a); Barker & Campbell (1981b); Howard et al. (1985); O'Callaghan et al. (1999); Schatz (1993)). If the protein is expressed in a soluble form in E. coli, these proteins are biotinylated by the host cell's native BirA enzyme. Alternatively, if another host organism is used or if the protein is expressed in inclusion bodies and refolded in vitro, the protein may be biotinylated in vitro using purified enzyme, Mg²⁺-ATP and biotin. Biotinylated proteins may simply be flowed over the flow-cell containing the immobilised streptavidin to give effective coupling of the biotinylated protein to the flow-cell surface.

Assaying for Specific Binding Between Soluble TCR and MHC-Flu-Peptide

The interactions between sTCR and its ligand/MHC complex or an irrelevant MHC-peptide combination (as described above) is analysed on a BIAcore 3000™ surface plasmon resonance (SPR) biosensor. SPR measures changes in refractive index expressed in response units (RU) near a sensor surface within a small flow cell, a principle that can be used to detect receptor ligand interactions and to analyse their affinity and kinetic parameters. The probe flow cells are prepared by immobilising the individual HLA-peptide complexes in separate flow cells via binding between the biotin cross linked onto β2 m and streptavidin which have been chemically cross linked to the activated surface of the flow cells. The assay is then performed by passing sTCR over the surfaces of the different flow cells at a constant flow rate, measuring the SPR response in doing so. Initially, the specificity of the interaction is verified by passing sTCR at a constant flow rate of 5 μl min⁻¹ over three different surfaces; one coated with ˜5000 RU of specific peptide-HLA complex, the second coated with ˜5000 RU of non-specific peptide-HLA complex. Injections of soluble sTCR at constant flow rate and different concentrations over the peptide-HLA complex are used to define the background resonance. The values of these control measurements are subtracted from the values obtained with specific peptide-HLA complex and are used to calculate binding affinities expressed as the dissociation constant, Kd.

### Example 15

### Primary Screening for Compounds Which Inhibit the Interaction of the T Cell Receptor with its Ligand Peptide-MHC Complex

Producing Recombinant sTCR and pMHC

Recombinant soluble T cell receptor (sTCR) and peptide-MHC complexes (pMHC) are made essentially as described in previous Examples, or using any other appropriate methods such as that described in U.S. Pat. No. 5,869,270 to produce single chain MHCs, except that specific tags may be engineered onto the C-terminus of both proteins e.g. hexa-histidine on the soluble TCR and biotinylation tag on the pMHC. The recombinant biotinylation tag may be specifically biotinylated in vitro using the bacterial enzyme BirA according to the method of O'Callaghan et al, (1999). Other known tags may also be utilised.

Small Molecule Compound Libraries

In the search for compounds which inhibit the interaction between sTCR and pMHC, attention is focussed on the identification of small, that is, low molecular weight, compounds with therapeutic potential. This is generally because such compounds: are usually inexpensive to produce; can often be relatively easily and swiftly modified so as to provide variants of a “lead compound” which may have different properties; are often relatively stable, or can be modified to be stable, in the body, in particular compared to proteins and other biochemical substances; are less likely to provoke unwanted physiological reactions, like immune responses, than larger entities; and are more likely to be able to be administered orally because they are more likely to be able to pass the membrane barriers of the digestive tract into the blood, while less likely to be degraded by the digestive system. Recent advances in combinatorial chemistry, enabling relatively easy and cost-efficient production of very large compound libraries, have increased the scope for compound testing enormously.

Primary High-Throughput Screening (HTS)

A primary screening assay is designed, in a high-throughput format, which can detect the interaction between the soluble TCR and the peptide-MHC complex. Such assays are well known and include such technologies as SPA (scintillation proximity assay), HTRF (homogeneous time-resolved fluorescence), the Alpha screen, and SPR (surface plasmon resonance). Such high-throughput assays allow the rapid testing of many compounds for their ability to inhibit the interaction measured. Compounds which give a validated decrease in the measured specific interaction are known as ‘hits’. Typically, the primary screen identifies (very) approximately 0.1-1% of the compounds tested as hits. This identification of hit compounds therefore comprises a major step in the process of identifying drug candidates from libraries of compounds.

Although such hit compounds demonstrate (usually repeated) inhibition in a primary screening assay, they do not always show genuine biological activity because of the possibility of artefacts in the screening assay. It is therefore necessary to further validate their activity using a secondary screen before the compound can become a lead compound.

### Example 16

### Secondary Screening for Compounds Which Inhibit the Binding of sTCR and pMHC Using Surface Plasmon Resonance (SPR)

FIG. 3 outlines a method for using SPR detection of TCR-MHC/peptide interactions to test, or screen for, compounds that inhibit or block the MHC/peptide surface for TCR binding.

Test ligand A=MHC/peptide complex for which a compound with binding specificity is sought.

Control ligand B=MHC/peptide complex with identical MHC but a different peptide.

Test receptor C=TCR which recognises test ligand A

Control receptor D=TCR which recognises control ligand B

Test compound E=test compound

The two MHC/peptide complexes A and B with identical MHC proteins but presenting different peptide antigens are produced as soluble molecules according to one of the methods described ((Garboczi et al (1992); Madden et al (1993); Garboczi et al (1994); Reid et al (1996); Smith et al (1996); Gao et al (1997); Gao et al Prot. Sci. 7: 1245-49 (1998); Kalandadze, et al. (1996); Hansen(1998); Frayser et al. (1999)), and immobilised in the respective sensor cells.

Soluble TCRs can be produced as described in WO99/60119, WO99/60120, Willcox et al, (1999a) and Willcox et al, (1999b).

Referring to FIG. 3d, if the test sample flowed over sensor cells 1 and 2 contains a compound E that binds with high stability to the MHC/peptide complex A in sensor cell 1, a higher constitutive level of readout may be observed if the compound E is of sufficient size for a change in mass to be detected. However, whether the compound E itself produces a sufficient change in mass for detection is immaterial, since the presence and specificity of the MHC/peptide-compound interaction is demonstrated by subsequent testing with the relevant and control TCRs (FIGS. 3e and 3f, respectively). With the compound E bound to the MHC/peptide complex A in sensor cell 1, the TCR C cannot bind but can still bind in sensor cell 3, which was not exposed to the compound test sample E (FIG. 3). This serves to demonstrate that the TCR C is functional and that lack of binding to sensor cell 1 is caused by the compound E. Normal binding of TCR B in sensor cell 2 demonstrates that the compound E has not bound here and is specific for the peptide of complex A (FIG. 3f).

It is important to note that the low affinities and fast kinetics of the TCR-MEC/peptide interaction are crucial to this screening strategy. Only because of the fast off-rates of TCR-MHC/peptide interactions (Willcox, et al. (1999a)), is binding detected only while the samples of soluble TCRs are flowed over the sensor surfaces. The MHC/peptide complex is left free to be bound by another compound almost immediately after the soluble TCR sample has flowed through the sensor cell.

The method could be modified by using four sensor cells instead of three. In this case, simultaneous screening could be performed for compounds with affinity for either MHC/peptide complex A or B. The sensor cell 4 would have MHC/peptide complex B immobilised therein and serve the equivalent control purposes for binding to sensor cell 2 as sensor cell 3 does for sensor cell 1. The two TCRs C and D would serve as specificity controls for each other.

The human body has the capacity to produce huge repertoires of two types of antigen receptors, antibodies (Ab's) and TCRs. Ab's and TCRs constitute the basis for adaptive immunity. Ab's bind suitable epitopes through interactions that are usually characterised by relatively high affinity. In contrast, TCR binding to MHC/peptide is characterised by low affinity, with recognition of the antigen presenting cell by the T cell relying on higher avidity accomplished through multiple interactions. This also appears to be the case for many other interactions between cell-surface proteins involved in regulating the cellular immune system (Davis, et al. (1998).

Three features of TCR recognition of MHC/peptide make this class of interactions particularly attractive for interference by small compounds:

TCRs are specific for cell-surface antigens. Thus, if a small compound is found that only binds to a particular MHC/peptide complex and interferes with TCR binding, then this compound must be peptide antigen-specific. Because humans of the same MHC type usually present the same peptide antigen when suffering from a particular disease (be it viral infection, cancer or immune disorder), such a compound will have specificity for the disease-relevant cells in the affected population of the relevant MHC type.

Because of the relatively low affinity of TCR-MHC/peptide interactions, there is a considerable range of affinities within which compounds with MHC/peptide binding specificity would have higher affinities than TCRs. There is thus considerable scope for identifying compounds that would be suitable as T cell inhibitors by means of competitive binding to MHC/peptide complexes.

TCR signalling is exquisitely sensitive to interference, as demonstrated by “T cell antagonism” in which subtly modified peptide ligands display great potency for preventing full signalling activation in response to the “normal’ peptide antigen (Klenerman, et al. (1995); Sloan et al (1995); Sloan et al. (1996); Sewell et al. (1997); Purbhoo et al (1998)). Thus, T cell responses may also be sensitive to interference by other means, for instance, interference by competitive ligand binding by small compounds.

These considerations make it likely that TCR-MHC/peptide interactions are suitable targets for T cell inhibition with small compounds. In humans, this type of therapy may be useful to prevent unwanted T cell responses, for example those causing autoimmune diseases or graft rejection following transplant operations. In particular, MHC/peptide-specific compounds are likely to be substantially more specific in their immune inhibitory effect than currently applied treatments for such conditions.

Compounds specific for peptide antigens presented on the cell surface as a consequence of, for example, viral infections or cancerous transformation of body cells also have therapeutic potential, albeit for different applications than immune inhibition. Such compounds could for instance be used as carriers of other, cytotoxic, compounds. Such compounds are well-known to the skilled person and include cis-platin, cytotoxic alkaloids, calcein acetoxymethyldester (Johsson et al, (1996)), and 5-fluoroorotate (Heath et al, (1985)). This strategy could be applied for highly specific drug delivery strategies in the human body. In some cases, most notably in cancer tumours, not all malignant cells present antigen, and it may be desirable to affect a local area rather than only the subset of cells that are antigen presenting. Cytotoxic T cells do not have this capacity but, depending on the therapeutic agent which is carried, it may be possible to achieve such an effect by in vivo drug delivery mediated by a small peptide antigen-specific compound.

In addition, peptide-specific compounds could have potential in diagnostics, for instance by coupling it to a biosensor, or in in vivo imaging by coupling it to a suitable detectable reagent. Such reagents are well-known to the skilled person and include Gd-containing liposomes (Trubetskoy et al, (1995)) and MION 46 (Shen et al, (1996)).

### REFERENCES

Alam et al., (1996). Nature 381:616-20

Altman et al., (1996) Science 274(5284): 94-6.

Baker et al., (2000). Immunity 13:475-8

Banks et al., (1993) J Virol 67: 613-616.

Barker & Campbell (1981a) J Mol Biol 1981 146:4 451-67

Barker & Campbell (1981b). J Mol Biol 1981 146:4 469-92

Broder & Earl (1999) Mol Biotechnol 13:223-45

Carmichael, (2000). In Lymphocytes A practical approach (ed S. L. Rowland-Jones and A. J. McMichael). Oxford University Press, UK.

Chakrabarti et al., (1985) Mol Cell Biol. (12):3403-9.

Chakraborty & Mukherji (1998). Cancer Res 58:1363-6

Chambers et al, (2000) J Pathol 192 (3) 280-288.

Chen & Platsoucas (1992) Scand J Immunol 35:539-49

Davis, et al., (1998). Annu. Rev. Immunol. 16: 523-544;

De Fazio, et al. (1996) Transplantation 61: 104-10

Ebers & Dyment (1998) Semin Neurol 18(3):295-9

Engelhard V H: (1994) Curr Opin Immunol 6: 13-23.

Falkner & Moss (1988) J. Virol. 62 (6):1849-54.

Feltkamp et al., (1993) Eur J Immunol 23: 2242-2249.

Fossum et al., (1994) Int J Cancer 56: 40-45.

Frayser et al. (1999) Protein Expr Purif 15: 105-14

Garboczi et al, (1996) J Immunol 157:12 5403-10

Garboczi et al, (1992) Proc Natl Acad Sci USA 89:8 3429-33

Garboczi et al (1994) J Mol Biol 239(4): 581-7

Gao et al., (1997) Nature 387:6633 630-4

Gao et al. (1998) Protein Sci 7(5):1245-9

Germain, (1994.) Cell. 76:287-299.

Hall & Otley, (1991) Semin Dermatol Immunogenetics of dermatitis herpetiformis 10(3):240-5

Hansen et al., (1998) Tissue Antigens 51 (2): 119-28

Heath et al, (1985) FEBS Lett. 187: 73-5

Henseler (1998) Arch Dermatol Res 290(9):463-76

Houbiers et al., (1993) Eur J Immunol 23: 2072-2077.

Howard et al. (1985) Gene 35 (3): 321-31

Jager et al., (2000). Proc Natl Acad Sci USA 97:4760-5

Janetzki et al., (2000). J Immunol Methods 234:1-12

Jenh et al, Anal Biochem. 165(2) 287-93

Johsson et al, (1996) Eur. J. Cancer. 32a 883-7

Kalandadze, et al. (1996) J Biol Chem 271: 20156-62

Kennedy (2000).EXS 89 1-10.

Khanna et al., (1993) J Immunol 150: 5154-5162.

Klenerman, et al. (1995) Eur J Immunol 25(7): 1927-31.

Lechler et al., (2000), HLA in Health and Disease. 2^{nd} Ed. Academic Press

Lopez-Larrea et al. (1998) Mol Med Today 4(12):540-9

Lozzio & Lozzio (1975) Blood 45: 321-334,

Luka, et al., (1978). J Virol., 27, 604.

Lyons et al., (1996). Immunity 5:53-61

Madden et al., (1993) Cell 1993 75: 4 693-708

Masucci (1993) Curr Opin Immunol 5: 693-700

Mazzara et al., (1993) Methods Enzymol 217:557-81

Moss et al., (1991) Proc Natl Acad Sci USA 88(20):8987-90

Nayersina et al., (1993) J Immunol 150: 4659-4671.

O'Callaghan et al. (1999) Anal Biochem 266 (1) 9-15

Ogg et al, (2000) Br J Cancer 2000 Mar 82:1058-62

Olivieri et al. (1998) Eur J Radiol 27 Suppl 1:S3-6

O'Shea et al, (1989) Science 1989 Aug 245:646-8

Pena et al, (1998) Scand J Gastroenterol Suppl. 225:56-8 (1998)

Purbhoo et al (1998) Proc. Natl. Acad. Sci. USA 95: 4527-4532

Rammensee et al., (1993) Annu Rev Immunol 11:213,

Reid et al (1996) FEBS Lett 383: 1-2 119-23

Rickinson et al., (1984) Cell Immunol. 87(2): 646-58.

Ridgway & Fathman (1998) Clin Immunol Immunopathol 86 (1):3-10

Rosa & Fellous (1982) C R Seances Acad Sci III Oct 11;295 (5):359-64

Sambrook & Russell, (2001) Molecular Cloning, a Laboratory Manual. Third edition, ISBN 0-87969-576-5

Schatz (1993) Biotechnology NY 11(10): 1138-43

Scott, (1994) Current Opinions in Biotechnology, 5 40-48

Sewell et al. (1997) Eur J Immunol 27 (9): 2323-9

Shen et al, (1996) Bioconjug. Chem. 7:3 311-6

Skipper & Stauss (1993) J Exp Med 177:5 1493-8

Slansky et al., (2000). Immunity 13:529-38.

Sloan et al, (1995) Curr Opin Immunol 7(1): 103-9

Sloan et al., (1996) Annu Rev Immunol 14: 1-27

Smith et al, (1996) Immunity 4:3 215-28

Tay-Kearney et al, (1996) Am J Ophthalmol 121(1):47-56

Tosato, G. (1997). In Current protocols in immunology (ed J. E. Coligan, A. M. Kruisbeek, D. H. Margulies, E. M. Shevach, and W. Strober), p7.22.1 Wiley, NY.

Trubetskoy et al, (1995) Magn. Reson, Imaging 13: 31-7

Van der Merwe et al, J. Exp. Med. 185:393-403 (1997)

Willcox et al, (1999a) Immunity 10: 357-365

Willcox et al., (1999b) Protein Sci 8:11 2418-23

Yoshida M. et al, (2001). Curr Pharm Des 7 (4) 291-310.

Zamani & Cassiman, (1998) Am J Med Genet. 76(2):183-94

1. A method for identifying an entity which binds to a peptide-Major Histocompatibility Complex (pMHC) complex, the method comprising:
complexing a peptide with a predetermined MHC thereby forming a pMHC complex, wherein the peptide is obtained from a protein sequence and is not known to be capable of being complexed with the predetermined MHC; and
screening for an entity which binds to the pMHC complex.

2. A method as claimed in claim 1, wherein the protein sequence is identified by proteomic analysis.

3. A method as claimed in claim 1, wherein the protein sequence is predicted from a nucleic acid sequence.

4. A method as claimed in claim 3, wherein the nucleic acid sequence is identified by genomic analysis.

5. A method as claimed in any preceding claim, wherein the peptide is taken from a population of peptides, all of which are obtained from the protein sequence.

6. A method as claimed in claim 5, wherein the population of peptides represents all or a part of the protein sequence.

7. A method as claimed in claim 6, wherein the protein sequence is modified relative to a native protein sequence, the population of peptides representing the part(s) of the protein sequence which is/are modified.

8. A method as claimed in claim 9 wherein the or each modification is caused by a mutation in the nucleic acid (e.g. DNA) sequence encoding the native protein sequence.

9. A method as claimed in any one of claims 5 to 8, wherein the population of peptides is a series of overlapping peptides.

10. A method as claimed in any preceding claim, wherein the peptide consists of the minimum sequence necessary to enable T cell receptor binding to the pMHC complex.

11. A method as claimed in any preceding claim, wherein the Major Histocompatibility Complex (MHC) is Human Leukocyte Antigen (HLA).

12. A method as claimed in claim 11, wherein the HLA is a class I class II or non-classical HLA molecule.

13. A method as claimed in any preceding claim, wherein the pMHC complex is produced by transforming cells presenting MHC molecules with a vector which expresses the protein sequence or peptide.

14. A method as claimed in claim 13, wherein the vector is a virus.

15. A method as claimed in claim 14, wherein the virus is a Vaccinia virus.

16. A method as claimed in any preceding claim, which further comprises the step of identifying whether the pMHC complex binds to a T cell receptor, the step comprising contacting the pMHC complex with T cells or with T cell receptors under conditions suitable to allow binding between the T cell receptors and the pMHC complex and identifying whether binding between the T cell receptors and the pMHC complex has occurred.

17. A method as claimed in claim 16, which further comprises the step of isolating a T cell which binds to the pMHC complex.

18. A method as claimed in any preceding claim, which comprises isolating a T cell receptor specific for the pMHC complex.

19. A method as claimed in claim 18 wherein the T cell receptor is isolated by isolating nucleic acid encoding the T cell receptor, transforming or transfecting a host cell with said nucleic acid, culturing the host cell under conditions enabling expression and isolating the expressed T cell receptor.

20. A method as claimed in any one of claims 16 to 19, wherein the T cell receptor is soluble T cell receptor.

21. A method as claimed in any preceding claim, further comprising screening for an entity which interferes with the interaction between the pMHC complex and a T cell or T cell receptor.

22. A method as claimed in claim 21, the method comprising contacting a candidate entity with a T cell receptor and the pMHC complex under conditions suitable to allow interaction between the candidate entity, the T cell receptor and the pMHC complex, and determining the extent of interference by the candidate entity to the interaction between the T cell receptor and the pMHC complex.

23. A method as claimed in any preceding claim, wherein the protein sequence is associated with one or more diseases.

24. A method as claimed in claim 23, wherein the disease is an auto-immune disease, a cancer-associated disease, transplant rejection or graft-versus-host disease.

25. A method as claimed in claim 24, wherein the auto-immune disease is one or more of Hashimoto's disease, rheumatoid arthritis, osteo arthritis, dermatitis, chronic active hepatitis, pemphigus vulgaris, systemic lupus erythermatosus, myasthenia gravis, coeliac disease, Sjogren's syndrome, Addison's disease, insulin-dependant diabetes, Grave's disease, primary myxedema, Goodpasture's syndrome, tuberculoid leprosy, multiple sclerosis, ankylosing spondylitis, Reiter's disease, arthritis (post-salmonella, post-shigella, post-yersinia or post-gonococcal), uveitis, amyloidosis in rheumatoid arthritis, thyroiditis, psoriasis vulgaris, or idiopathic hemochromatosis.

26. A method as claimed in claim 24, wherein the cancer-associated disease is one or more of breast cancer, colon cancer, skin cancer, ovarian cancer, leukaemia lymphoma, lung cancer, liver cancer, testicular cancer and nasophangeal malignancies.

27. A process Which comprises two or more methods as set out in any preceding claim comprising two or more peptides as set out in any preceding claim, and wherein each method comprises a different peptide in the pMHC.

28. An entity identifiable by the method as claimed in any one of claims 1 to 25 or by the process as claimed in claim 27.

29. The use of a pMHC complex, which complex binds to a T cell receptor, to identify a molecule which binds to the pMHC complex.

30. A method as substantially hereinbefore described with reference to, and/or as shown in, FIG. 1 and in the accompanying Examples.

31. A process as substantially hereinbefore described with reference to, and as shown in, the accompanying Examples.

32. A use as substantially hereinbefore described with reference to, and as shown in, the accompanying Examples.
